(19) United States
(12) Patent Application Publication
(10) Pub. No.: US 2003/0130514 A1
(43) Pub. Date: Jul. 10, 2003

(54) Preparation of alpha-diimines

(76) Inventor: Alex Sergey Ionkin, Kennett Square, PA (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/334,022

(22) Filed: Dec. 30, 2002

Related U.S. Application Data
(60) Provisional application No. 60/343,927, filed on Dec. 28, 2001.

(51) Int. Cl. C 07D 4 3/02; C07C 251/08

(52) U.S. Cl. 544/405; 564/271

(57) ABSTRACT

Alpha-diimines are prepared from anilines and alpha-ketals such as tetramethoxypropane in the presence of an acidic catalyst.

### FIELD OF INVENTION

The invention relates to a method of preparing alpha-diimines from amines and alpha-ketals in the presence of an acidic catalyst.

### BACKGROUND

Alpha-diimines are very versatile compounds and can be used as intermediates for other di-substituted compounds containing nitrogen function groups. They are also useful as ligands in catalysis and as polymer modifiers. They can be, however, difficult to prepare in large yields. A common preparation is via the addition of amines to ketones or aldehydes. Various substituted diones have been reacted with anilines to produce alpha-diimines (U.S. Pat. No. 6,015,851, Van Asselt, R. et. al, Recl. Trav. Chim. Pays-Bas, 113, p88-98, 1994). Diiminosuccinonitrile was prepared via the oxidation of diaminomaleonitrile (U.S. Pat. No. 3,862,205).

The reaction of o-phenylenediamine with 1,1,3,3-tetramethoxypropane in the presence of a nickel salt yielded a delocalized dibenzotetrazaannulene complex, but no diimine was observed. (Cutler, A., et al., J. Coord. Chem., 6, p59-61, 1976).

### SUMMARY OF THE INVENTION

The invention is directed to a process to prepare diimines of Formula 1:

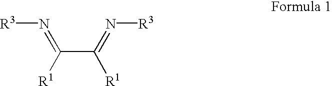

by contacting a compound of Formula 2:

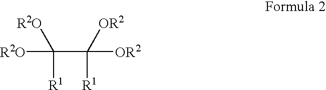

with an amine of the formula R³NH₂ in the presence of an acidic catalyst, wherein R¹ and R² are independently an optionally substituted C1 to C6 alkyl or aryl; and R³ is an optionally substituted C1 to C6 alkyl, aryl or heteroaryl. Preferably R¹ and R² are independently C1 to C3 alkyl, and R³ is an optionally substituted phenyl. More preferably R¹ and R² are methyl.

A preferred acidic catalyst is selected from the group consisting of Lewis acids, inorganic acids, organic sulfonic acids, organic phosphoric acids, heteropolyacids, perfluoroalkyl sulfonic acids, their metal salts, and mixtures thereof. More preferred is where the acidic catalyst is a metal halide, organic sulfonic acid or organic phosphoric acid; most preferred is where the acidic catalyst is p-toluenesulfonic or boron trifluoride.

### DETAILED DESCRIPTION OF THE INVENTION

The invention comprises a process to prepare diimines of Formula 1:

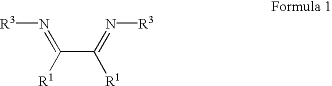

by contacting a compound of Formula 2:

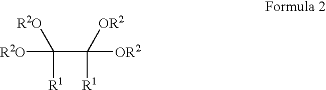

with an amine of the formula R³NH₂ in the presence of an acidic catalyst, where R¹ and R² are independently an optionally substituted C1 to C6 alkyl or aryl, and R³ is an optionally substituted C1 to C6 alkyl, aryl or heteroaryl. Preferably R¹ and R² are independently C1 to C3 alkyl, and R³ is an optionally substituted phenyl. More preferably R¹ and R² are methyl.

“Alkyl” means an alkyl group up to and including 6 carbons. They can be linear or cyclic. Common examples of such alkyl groups include methyl, ethyl, propyl, isopropyl, butyl, s-butyl, isobutyl, pentyl, neopentyl, hexyl, and cyclohexyl.

“Aryl” means a group defined as a monovalent radical formed conceptually by removal of a hydrogen atom from a hydrocarbon that is structurally composed entirely of one or more benzene rings. Common examples of such hydrocarbons include benzene, biphenyl, terphenyl, naphthalene, phenyl naphthalene, and naphthylbenzene.

“Heteroaryl” refers to unsaturated rings of 5 or 6 atoms containing one or two O and S atoms and/or one to four N atoms provided that the total number of hetero atoms in the ring is 4 or less, or bicyclic rings wherein the five or six membered ring containing O, S, and N atoms as defined above is fused to a benzene or pyridyl ring. Common examples are furan and thiphene.

“Substituted” means a group that is substituted and contains one or more substituent groups that do not cause the compound to be unstable or unsuitable for the use or reaction intended. Substituent groups which are generally useful include nitrile, ether, ester, halo, amino (including primary, secondary and tertiary amino), hydroxy, oxo, vinylidene or substituted vinylidene, silyl or substituted silyl, nitro, nitroso, sulfinyl, sulfonyl, sulfonic acid alkali metal salt, boranyl or substituted boranyl, and thioether.

A suitable acidic catalyst can be defined either as a substance which has the ability to donate protons as defined by Brönsted, or as a substance which can form a covalent bond with an atom, molecule or ion that has an unshared electron pair as defined by Lewis. A further definition of acid catalysts and how to determine if a particular substance is acidic is explained in Tanabe, K., Catalysis: Science and Technology, Vol. 2, pg. 232-273, ed. Anderson, J. and Boudart, M., Springer-Verlag, N.Y., 1981.

Suitable Brönsted acids are those with a pKa less than about 4, preferably with a pKa less than about 2. They can include inorganic acids, organic sulfonic acids, organic phosphoric acid, heteropolyacids, perfluoro-alkyl sulfonic acids and mixtures thereof. Also suitable are metal salts of acids with a pKa less than about 4, including metal sulfonates, metal trifluoroacetates, metal triflates, and mixtures thereof including mixtures of the salts with their conjugate acids. Specific examples of catalysts include sulfuric acid, fluorosulfonic acid, phosphoric acid, phenylphosphonic acid, p-toluenesulfonic acid, benzenesulfonic acid, phosphotungstic acid, phosphomolybdic acid, trifluoromethanesulfonic acid, methanesulfonic acid, p-toluicsulfonic acid, 1,1,2,2-tetrafluoroethanesulfonic acid, 1,1,1,2,3,3-hexafluoropropanesulfonic acid, and triflatic acid and its salts. Sulfonic acids are compounds with at least one —SO2H group or its salt. Examples of preferred sulfonic acids include methanesulfonic acid, toluenesulfonic acid, and benzenesulfonic acid. A preferred catalyst is an organic sulfonic or organic phosphoric acid; more preferred is p-toluenesulfonic acid.

Suitable Lewis acid catalysts, include metal halides such as aluminum bromide, aluminum chloride, boron trifluoride, boron trichloride, boron tribromide, titanium tetrachloride, titanium tetrabromide, stannic chloride, stannic bromide, bismuth trichloride, and ferric chloride. A preferred catalyst is boron trifluoride, optionally used as a salt or as an etherate, such as boron trifluoride ethyletherate.

The process can be run in any solvent or any mixture of solvents, provided that the solvent is not detrimental to catalyst, reactant and product. One or more of the solvents may also be chosen from one of the reactants.

The process is preferably run at reflux temperatures and under an inert atmosphere such as nitrogen to avoid possible hydrolysis of the reagents. Reaction time can vary depending upon desired yield.

The process of the instant invention may additionally comprise the recovery or isolation of one or more of the unsaturated nitriles. This can be done by any method known in the art, such as distillation, decantation, recrystallization, chromatographic separation, or extraction. A preferred method is recrystallization.

### Materials and Methods

Unless otherwise specified, all compounds were purchased from Aldrich Chemical Co., Milwaukee, Wis.

X-ray analysis data collection was performed as follows:

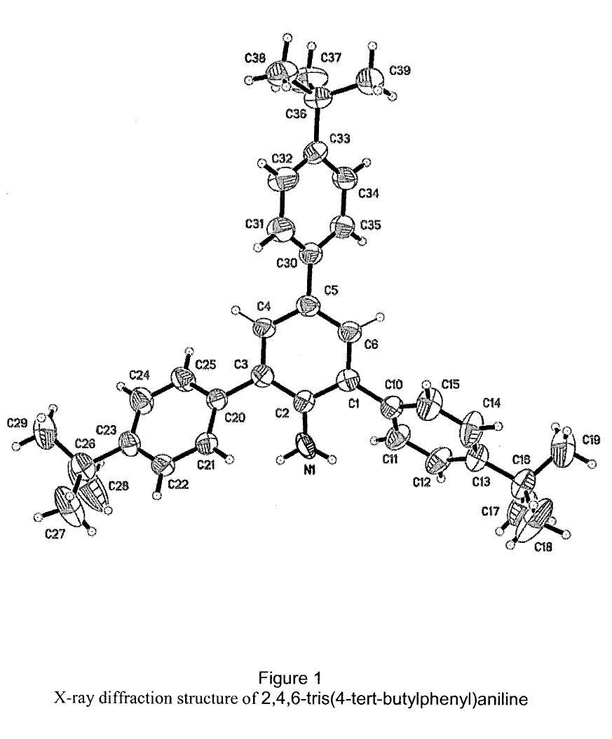

FIG. 1—Bruker SMART 1K CCD system, MoK alpha radiation, standard focus tube, anode power=50 kV×40 mA, crystal to plate distance=4.9 mm, 512×512 pixels/frame, multirun data acquisition, total scans=9, total frames=6000, oscillation/frame=−0.30°, exposure/frame=30.0 sec/frame, maximum detector swing angle=−40.0°, beam center=(255.50,253.00), in plane spot width=1.56, omega half width=6.49, SAINT integration, hkl min/max=(−30, 29, −10, 10, −36, 37), data input to shelx=81673, unique data=5913, two-theta range=2.92 to 56.62°, completeness to two-theta 56.62=99.60%, R(int-xl)=0.0544, SADABS correction applied.

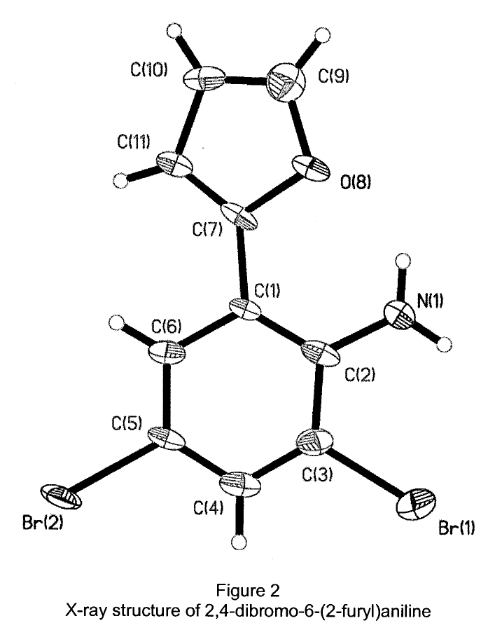

FIG. 2—X-ray analysis data collection was performed as follows: Bruker SMART 1K CCD system, MoKalpha radiation, standard focus tube, anode power=50 kV×40 mA, crystal to plate distance=4.9 mm, 512×512 pixels/frame, hemisphere data acquisition, total scans=4, total frames=1330, oscillation/frame=−0.30°, exposure/frame=40.0 sec/frame, maximum detector swing angle=−28.0°, beam center=(255.50,253.00), in plane spot width=1.60, omega half width=1.31, SAINT integration, hkl min/max=(−16, 15, −5, 5, −27, 27), data input to shelx=25074, unique data=2505, two-theta range=3.40 to 56.54°, completeness to two-theta 56.54=99.70%, R(int-xl)=0.0706, SADABS correction applied.

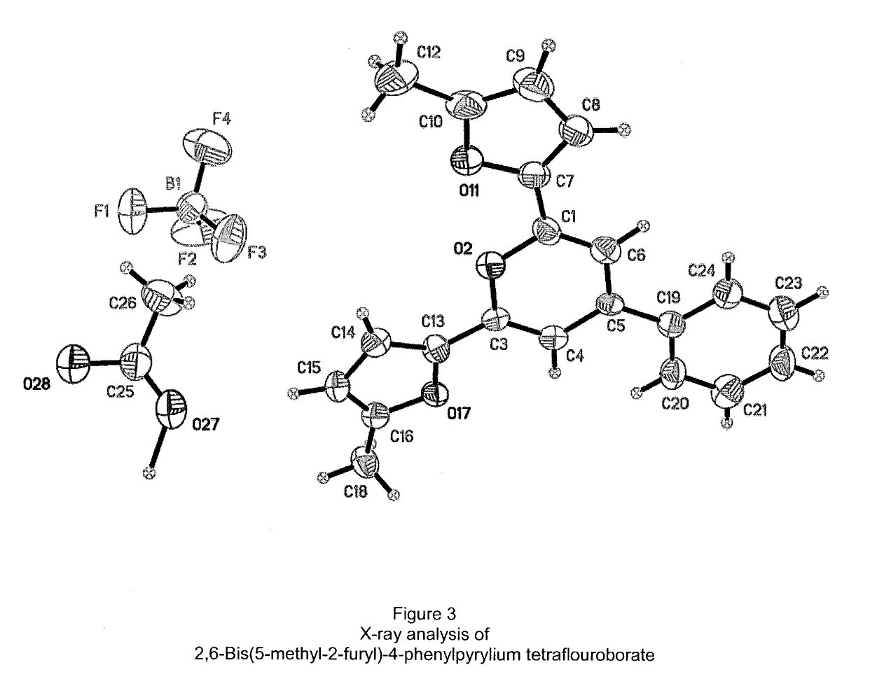

FIG. 3: Bruker SMART 1K CCD system, MoKalpha radiation, standard focus tube, anode power=50 kV×40 mA, crystal to plate distance=4.9 mm, 512×512 pixels/frame, hemisphere data acquisition, total scans=4, total frames=1330, oscillation/frame=−0.30°, exposure/frame=40.0 sec/frame, maximum detector swing angle=−28.0°, beam center=(255.50,253.00), in plane spot width=1.60, omega half width=0.83, SAINT integration, hkl min/max=(−8, 8, −14, 11, −23, 23), data input to shelx=8970, unique data=3007, two-theta range=3.54 to 45.96°, completeness to two-theta 45.96=99.70%, R(int-xl)=0.1162, SADABS correction applied.

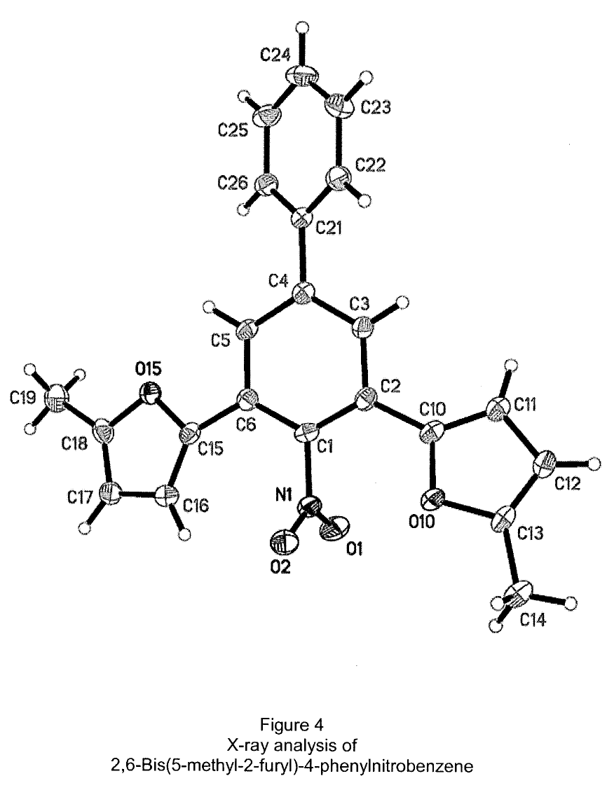

FIG. 4: Mar-CCD area detector, Synchrotron radiation, APS DND-CAT, Hutch 5ID-B, wavelength=0.7100, DENZO integration for single scan, total frames collected=60, scan width per frame=3.00°, exposure time pre frame=4.00 seconds, xtal-detector distance=54.70 mm, Total data collected 24363, Rmerge from DENZO=0.0470, hkl min/max=(0, 9, 0, 28, −25, 25), data input to shelx=6800, unique data=6800, two-theta range=5.88 to 52.78°, completeness to two-theta 52.78=97.20%, R(int-xl)=0.0000,

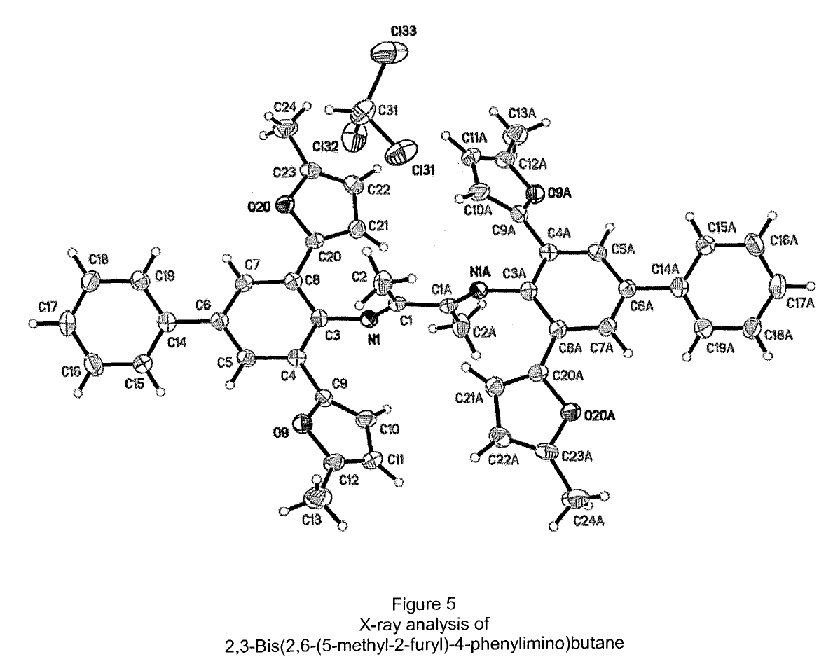

FIG. 5: Bruker SMART 1K CCD system, MoKalpha radiation, standard focus tube, anode power=50 kV×40 mA, crystal to plate distance=4.9 mm, 512×512 pixels/frame, hemisphere data acquisition, total scans=4, total frames=1330, oscillation/frame=−0.30°, exposure/frame=8.0 sec/frame, maximum detector swing angle=−28.0°, beam center=(255.50,253.00), in plane spot width=1.29, omega half width=0.86, SAINT integration, hkl min/max=(−10, 11, −19, 16, −22, 24), data input to shelx=11389, unique data=5268, two-theta range=3.58 to 56.56°, completeness to two-theta 56.56=93.50%, R(int-xl)=0.0427, SADABS correction applied.

### EXAMPLE 1

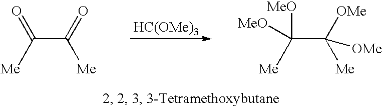

65 ml of 2,3-butanedione (0.748 mole), 190 ml of trimethyl orthoformate (1.739 mole), 50 ml of methanol and a few drops of sulfuric acid were refluxed 20 hours under argon. NaHCO₃ as powder (2.2 g) was added to the cool reaction mixture. Concentration under reduced pressure afforded an orange liquid that was diluted with ether and washed with saturated aqueous NaHCO₃ two times. A clear yellow liquid was obtained after concentration. This material was distilled to afford a colorless liquid (94.5 g, 71%) with boiling pt. 45-50° C. (0.5 mm Hg). Lit. boiling pt. 172-174 (760 mm Hg) found in J.-L. Montchamp, F. et. al, J.Org. Chem., 61,#11, 3897-3899,1996. ¹H NMR (CDCl₃) 0.85 (s, 6H, CH₃), 2.89 (s, 12H, MeO). ¹³C NMR (CDCl₃) 18.75 (CH₃), 49.05 (MeO), 102.87 (C(CH₃)(OMe)).

### EXAMPLE 2

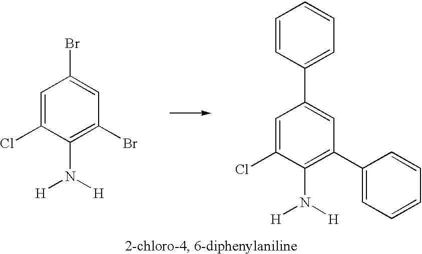

5.0 g (0.01752 mole) of 2-chloro-4,6-dibromoaniline, 4.7 g (0.0386 mole) of phenylboronic acid, 17.0 g (0.0482 mole) of cesium carbonate, 0.89 g (0.00077 mole) of tetrakis(triphenylphosphine)palladium and 120 ml of dioxane were refluxed for 24 hours under argon. The reaction mixture was filtered and the solvent was removed under vacuum. The resulted mixture was purified by chromatography on silica with eluent petroleum ether/ethyl ether as 10/2. Yield of 2-chloro-4,6-diphenylaniline was 2.01 g (41.0%) with m.p. 83.02° C. Elemental analysis calculated for C₁₈H₁₄ClN % C, 77.21, % H, 5.00, % N, 5.00; found % C, 77.69, % H, 4.97, % N, 4.87. LC/MS MW was 279.

### EXAMPLE 3

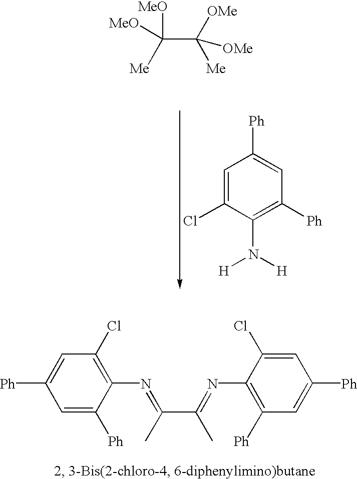

0.8 g (0.00285 mole) of 2-chloro-4,6-diphenylaniline from Ex. 2, 0.25 g (0.0014 mole) of 2,2,3,3-tetramethoxybutane from Ex. 1, 3 ml of toluene and one drop of boron trifluoride ethyl etherate were refluxed under nitrogen for 5 hours. The solvent and formed methanol were removed in vacuum. The resulting yellow solid was dissolved in ethyl ether to remove the products of condensation of boron trifluoride ethyl etherate. The ether solution was stripped and residue was recrystallized from pentane/methanol (1:1) at −15° C. Yield of the diimine was 0.54 g (63%) with m.p.152.83° C. LS/MS MW was 608 with two atoms of chloride in MW. ¹H NMR (CDCl₃) 1.75 (s, 6H, CH₃), 7.01-7.5 (broad lines, 24H, aromatic protons). ¹³C NMR (CDCl₃) 169.63 ppm (C═N bonds). Analysis for C₄₀H₃₀Cl₂N₂ calculated: C,78.74; H,4.92; N,4.59. Found: C, 79.07; H,4.86; N,4.35.

### EXAMPLE 4

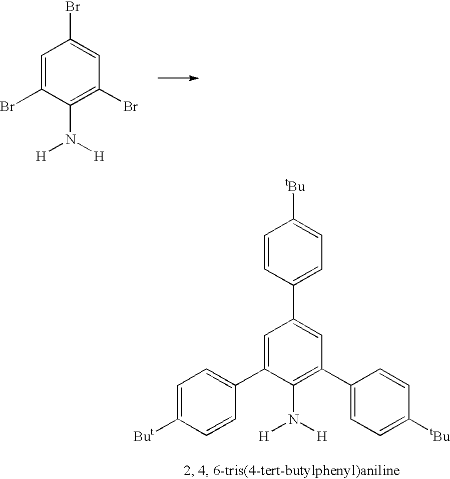

10.0 g (0.05616 mole) of para-tert-butylphenylboronic acid, 4.63 g (0.014 mole) of 2,4,6-tribromoaniline, 20.91 g (0.0642 mole) of cesium carbonate, 3.25 g (0.0028 mole) of tetrakis(triphenylphosphine)palladium and 120 ml of dioxane were refluxed for 24 hours under argon. The reaction mixture was filtered and the solvent was removed under vacuum. The resulted mixture was purified by chromatography on silica with eluent petroleum ether/ethyl ether as 10/2. Yield of 2,4,6-tris(4-tert-butylphenyl)aniline was 3.44 g (50.0%) with m.p. 205.69° C. Elemental analysis for calculated for C₃₆H₄₃N % C, 88.21, % H, 8.78, % N, 2.86; found % C, 88.47, % H, 8.85, % N, 2.91. LC/MS MW was 490 (M+H). The structure was proved by X-ray analysis, shown in FIG. 1.

### EXAMPLE 5

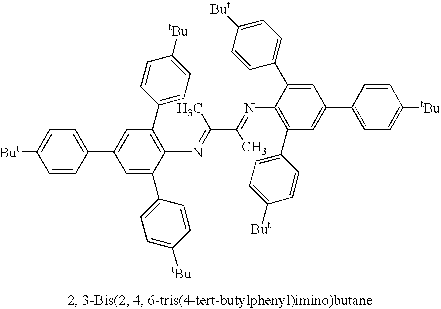

1.78 g (0.0036 mole) of 2,4,6-tris(4-tert-butylphenyl)aniline from Ex. 4, 0.29 g (0.0016 mole) of 2,2,3,3-tetramethoxybutane from Ex. 1, 20 ml of toluene and a few crystals of para-toluenesulfonic acid were refluxed under nitrogen for 26 hours. The solvent and formed methanol were removed in vacuum. The resulting yellow solid was recrystallized from ethanol. Yield of the diimine was 0.65 g (35%) with m.p. 333.87° C. (decomposition). ¹H NMR (CDCl₃) 1.11 (s, 36H, tBu), 1.20 (s, 9H, tBu), 1.22 (s, 6H, Me) 6.90-7.5 (broad lines, 28H, aromatic protons). ¹³C NMR (CDCl₃) 17.60(CH₃), 31.25(tBu), 34.33(tBu), 124.56, 125,50, 125.57, 125.83, 126.23, 127.74, 127.96, 128.71, 128, 78, 131.95, 136.48, 137.28, 137.43, 144.39, 149.45, 149.86 (aromatic carbons), 167.80 ppm (C═N bonds). Analysis for C₇₆H₈₈N₂ calculated: C,88.58; H, 8.55; N, 2.72. Found: C, 87.99; H, 8.87; N, 2.67.

### EXAMPLE 6

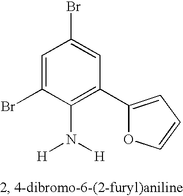

10.0 g (0.0893 mole) of 2-furanboronic acid, 7.37 g (0.0223 mole) of 2,4,6-tribromoaniline, 37.84 g (0.116 mole) of cesium carbonate, 5.16 g (0.000447 mole) of tetrakis(triphenylphosphine)palladium and 120 ml of dioxane were refluxed for 24 hours under argon. The reaction mixture was filtered and the solvent was removed under vacuum. The resulted mixture was purified by chromatography on silica with eluent petroleum ether/ethyl ether as 10/2. Yield of 2,4-dibromo-6-(2-furyl)aniline was 2.05 g (28.9%) with m.p. 62.19C. Elemental analysis for calculated for C₁₀H₇Br₂NO % C, 37.86, % H, 2.21, % N, 4.42; found % C, 37.75, % H, 2.04, % N, 4.18. The structure was proved by X-ray analysis, shown in FIG. 2.

### EXAMPLE 7

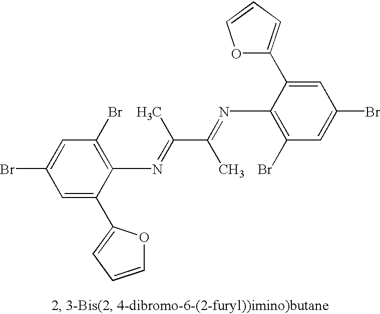

1.68 g (0.0053 mole) of 2,4-dibromo-6-(2-furyl)aniline from Ex. 6, 0.47 g (0.0026 mole) of 2,2,3,3-tetramethoxybutanefrom Ex. 1, 20 ml of toluene and a few crystals of para-toluenesulfonic acid were refluxed under nitrogen for 27 hours. The solvent and formed methanol were removed in vacuum. Resulted yellow solid was recrystallized from ethanol. Yield of the diimine was 0.23 g (12.71%) with m.p.347.07° C. (decomposition). ¹³C NMR (assignment only for selected bonds due to complexity of spectra) (CDCl₃)) 171.44 ppm (C═N bonds). Analysis for C₂₄H₁₆Br₄N₂O₂ calculated: N, 4.09. Found: N, 4.10. MALDI mass spec analysis was 685 (M+H).

### EXAMPLE 8

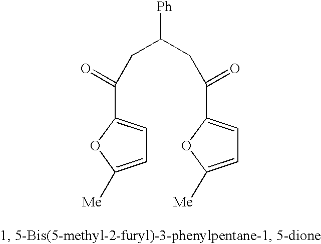

5.3 g (0.05 mole) of benzaldehyde, 18.60 g (0.15 mole) of 2-acetyl-5-methylfuran, 2.07 g (0.038 mole) of sodium methylate and 50 ml of dry methanol were stirred at room temperature for 3 days. The precipitate was filtered and recrystallized from ethanol. The yield of 1,5-bis(5-methyl-2-furyl)-3-phenylpentane-1,5-dione was 11.29 g (67%) with m.p.110.63C. ¹H NMR (CDCl₃) 2.34 (s, 6H, Me), 3.20 (m, 4H, CH2), 3.95 (m, 1H, CH), 6.1-7.49 (broad lines, 9H, aromatic and furyl protons). ¹³C NMR (assignment only for selected bonds due to complexity of spectra), (CDCl₃)) 186.73 ppm (C═O bonds). LC/MS MW was 337 (M+H).

### EXAMPLE 9

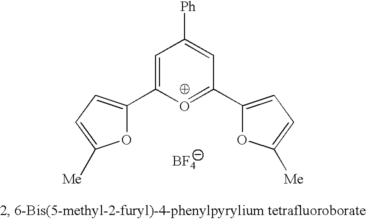

2.6 g (0.0077 mole) of 1,5-bis(5-methyl-2-furyl)-3-phenylpentane-1,5-dione from Ex. 8, 3.06 g (0.0093 mole) of triphenylcarbenium tetrafluoroborate and 20 ml of glacial acetic acid were refluxed for 2 hours. The reaction mixture was allowed to cool to ambient temperature and diluted with 200 ml of ethyl ether. The precipitate was collected and recrystallized from acetic acid. Yield of the pyrylium salt was 0.89 g (28.6%) with m.p. 90.62° C. The structure was proved by X-ray analysis, shown in FIG. 3.

### EXAMPLE 10

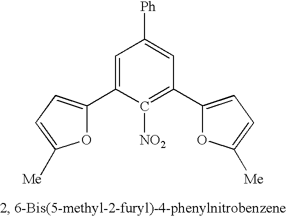

0.75 g (0.00185 mole) of 2,6-bis(5-methyl-2-furyl)-4-phenylpyrylium tetrafluoroborate from Ex. 10, 2.0 g (0.033 mole) of nitromethane, 2.0 g (0.020 mole) of triethylamine and 2 ml of ethyl alcohol were stirred at ambient temperature for 3 days. The solvent was removed in vacuum (0.1 mm) at room temperature and residue was purified by chromatography on silica with eluent petroleum ether/ethyl ether as 10/2. Yield of 2,6-bis(5-methyl-2-furyl)-4-phenylnitrobenzene was 0.37 g (56.1%) with m.p. 106.90° C. Elemental analysis for calculated for C₂₂H₁₇NO₄ % C, 73.46, % H, 4.73, % N, 3.90; found % C, 73.10, % H, 4.70, % N, 3.80. ¹H NMR (CDCl₃) 2.30 (s, 6H, Me), 6.01 (s, 2H, H-furyl), 6.51 (s, 2H, H-furyl) 7.20-7.75 (broad lines, 7H, aromatic protons). ¹³C NMR (CDCl₃) 13.45 (CH3),108.35, 110.44, 123.72, 123.81, 127.25, 128.30, 128.90, 139.29, 141.72, 142.93, 145.51, 153.82 (aromatic and furyl carbons). The structure was proved by X-ray analysis, shown in FIG. 4.

### EXAMPLE 11

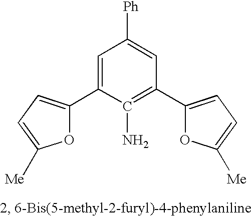

1.84 g (0.00512 mole) of 2,6-bis(5-methyl-2-furyl)-4-phenylnitrobenzene from Ex. 11, 5.0 g (0.077 mole) of zinc dust and 70 ml of glacial acetic acid were stirred at room temperature for 24 hours. The reaction mixture was filtrated and liquid part was washed with water and extracted with ethyl ether. After removal of the solvent, the residue was purified by chromatography on silica with eluent petroleum ether/ethyl ether as 10/2. Yield of 2,6-bis(5-methyl-2-furyl)-4-phenylaniline was 0.79 g (46.9%) with m.p. 54.38C. Elemental analysis for calculated for C₂₂H₁₉NO₂ % C, 80.15, % H, 5.77, % N, 4.25; found % C, 80.37, % H, 5.82, % N, 4.16. ¹H NMR (CDCl₃) 2.31 (s, 6H, Me), 6.03 (s, 2H, H-furyl), 6.50 (s, 2H, H-furyl) 7.19-7.65 (broad lines, 7H, aromatic protons). ¹³C NMR (CDCl₃) 14.14 (CH3), 107.81, 108.81, 118.46, 126.12, 126.87, 126.96, 129.04, 131.42, 139.96, 141.33, 151.56, 151.94 (aromatic and furyl carbons). LC/MS MW was 330 (M+H).

### EXAMPLE 12

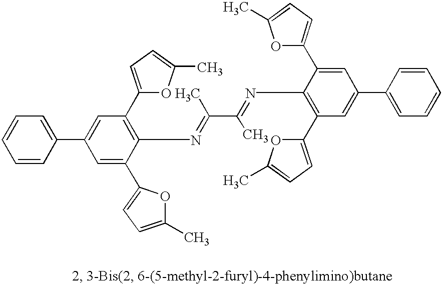

0.79 g (0.0024 mole) of 2,6-bis(5-methyl-2-furyl)-4-phenylaniline from Ex. 11, 0.21 g (0.0011 mole) of 2,2,3,3-tetramethoxybutane from Ex. 1, 20 ml of toluene and a few crystals of para-toluenesulfonic acid were refluxed under nitrogen for 18 hours. The solvent and formed methanol was removed in vacuum. Resulted yellow solid was recrystallized from ethanol. Yield of the diimine was 0.47 g (55.29%) with m.p.309.54° C. (decomposition). ¹³C NMR (assignment only for selected bonds due to complexity of spectra) (CDCl₃)) 170.55 ppm (C═N bonds). Analysis for C₄₈H₄₀N₂O₄ calculated: N, 3.95. Found: N, 3.59. MALDI mass spec analysis was 709 (M+H). The structure was proved by X-ray analysis shown in FIG. 5:

What is claimed is:

1. A process to prepare diimines of Formula 1: 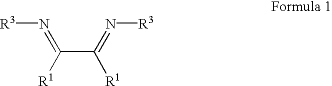
by contacting a compound of Formula 2: 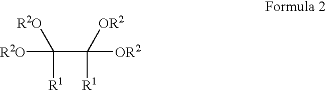
with an amine of the formula R³NH₂ in the presence of an acidic catalyst, wherein R¹ and R² are independently an optionally substituted C1 to C6 alkyl or aryl; and R³ is an optionally substituted C1 to C6 alkyl, aryl or heteroaryl.

2. The process of claim 1 wherein the acidic catalyst is selected from the group consisting of Lewis acids, inorganic acids, organic sulfonic acids, organic phosphoric acid, heteropolyacids, perfluoroalkyl sulfonic acids, their metal salts, and mixtures thereof.

3. The process of claim 2 wherein the acidic catalyst is a metal halide, organic sulfonic acid, or organic phosphoric acid.

4. The process of claim 3 wherein the acidic catalyst is p-toluenesulfonic or boron trifluoride.

5. The process of claim 1 wherein R¹ and R² are independently C1 to C3 alkyl, and R³ is an optionally substituted phenyl.

6. The process of claim 5 wherein R¹ and R² are methyl.
